SEQUENCE LISTING (6 sequences)

SEQ ID NO: 1 - protein, 381 aa, synthetic construct
  source 1..381: note = Amino acid sequence of PorV
MNLKSKLFVGLGLGLSLSAAAQEVKPVLTGAPFLRITPDARAAGMGDQGVATSTDAFSQFWNAAKYPFSKTTSAVAVNYTPYLSKLTSDVFLLYASYHQFLGQEERSTISASINYFNMGEVELNQLSGTQIVTTGKVKPNEFSVDVAYGLKLSDVYSMAVTGRYIRSDFGTFGDNSTIKPANTFAVDVSGFYQSEKHSSFGDYEGTARAGWAIQNLGPKLDYSGDENSRSYLPTTLRLGAGYDLYLDDMNKVGLSLEASKLLVPGSQLNNSDPANPKYEVPNVGVVEGIGKSFSNKKSVMLSGAVEYSYDDAFAVRGGYFHESDEQGGRRFATVGVGFKYQSFGLDISYLINTSKTNTALDNTLRFGLTWNIGGESSNANN

SEQ ID NO: 2 - protein, 156 aa, synthetic construct
  source 1..156: note = Amino acid sequence of Lipocalin
MKNLFLIGALGSALAVASCSPTKIAQNKKNKTDFAKLKGNWQITSVDYDHNYRVKPFDEGIDAQCFVGSQWKLVPNNYSGTYALLGEGRCPSFSRVIKFELVDGAEFKFKKIVEGTKAKDNIAGYSLYLLDQTDTSFSLEQNVSGVRIVYNFQKTN

SEQ ID NO: 3 - protein, 209 aa, synthetic construct
  source 1..209: note = Amino acid sequence of beta-barrel
MTKLISTALIGLSALVSAQVSFAGKAHLLFPTSSGSWKSLKDAGLKAYDKKGSNSVGYNVGVSAKVDLPGAFYVMPELYFTSFKNEFTDENTKTSLKAKSNRIDVPVLVGYKLLGDTASLFLGPVASYNLSTKESYQNFKEHATKDFTVGYQFGAQVQLSDFIINARYEGAFSNDQRNFINKHVVSGQEYEVQYDARPSLFMVGVGYKF

SEQ ID NO: 4 - DNA, 1146 bp, synthetic construct
  source 1..1146: mol_type = other DNA; note = Nucleotide sequence of gene encoding the PorV
atgaacttaaaatcaaaattatttgtaggacttgggcttgggcttagcctttcggcagcagctcaagaagtaaaacccgtacttacaggggctcctttcctcagaataactccagatgcaagagcagcaggtatgggggaccaaggggtagcaacctctacagatgcattctctcagttttggaatgccgctaagtacccattcagtaaaactacctctgctgtagcagtaaactatacgccttatctcagcaaacttacaagcgatgtattcctcctctatgcttcttatcatcagtttctggggcaggaagagcgatctaccatctctgcaagtatcaattattttaatatgggagaggtggaactcaatcaactgtcaggcacacaaatcgtaactacaggtaaggtaaagcctaatgaattttctgtagatgtagcctatgggctgaaactctcagatgtatactctatggcagttacaggtagatacatccgctcggatttcggtacctttggagacaacagcactataaaacctgccaatactttcgcagtagatgtttcaggtttctatcagtctgaaaagcactccagctttggagactacgaaggtacagcaagagcgggctgggctattcagaaccttggaccaaaattggactattcaggagatgaaaactctcgttcctatctgccaactacactcagattaggtgctggctatgatttatatctcgatgatatgaataaagtagggctttcccttgaagcatcaaaactcctcgttccaggctctcagctaaataattccgatcctgccaaccctaaatacgaagttcctaatgtaggtgtagtagagggtataggtaaatctttcagcaataaaaaaagcgtaatgctaagcggtgctgtggagtactcctatgatgatgcttttgctgtgcgtggtggttacttccacgagagcgatgagcagggtggcagacgcttcgctaccgtaggggtgggctttaaatatcagtctttcgggctggatatctcctacctcatcaatacttcaaagacaaatactgctcttgacaataccctccgtttcggtcttacctggaatattggtggtgagtcttccaacgccaataattag

SEQ ID NO: 5 - DNA, 471 bp, synthetic construct
  source 1..471: mol_type = other DNA; note = Nucleotide sequence of gene encoding the Lipocalin
atgaaaaacttatttctaataggagctttgggctctgcgttggcggtagcatcttgctcgcctacaaaaatagcacagaataaaaagaataagacagactttgcaaagctcaaaggcaattggcagattacaagcgtggattatgaccataattatcgtgtaaaaccttttgatgaaggcattgatgcgcagtgcttcgtcggtagtcaatggaaattggtgcccaataactactctggtacctatgcgctccttggtgaaggccgttgcccatcgttttccagagttataaagtttgagcttgtggatggtgcagagtttaagtttaaaaaaatagtagagggaaccaaggctaaggacaatatagcaggatactctctctatctcctcgaccaaaccgatacttcctttagcttagaacagaatgtaagcggtgtaagaatcgtttataacttccaaaagacaaattag

SEQ ID NO: 6 - DNA, 591 bp, synthetic construct
  source 1..591: mol_type = other DNA; note = Nucleotide sequence of gene encoding the beta-barrel
atgagaaagattattcttggagctatagtattaggagcaggattattctccaatgctaatgcacaaatccaaaaaggtaatttgataattggtggagacatcttcggtgctaattttggactgaacaaaggtggaggatataacttcaatcttcaaccgaaagcagcttattttgtagaagataatgtagcagtcggtggatatgcagacttaggtttcagtggctcgaacggaggacaaacaaaattcacttatacagtaggagctttgggaagatattacttatcaccaggacaaggtggcgtagataaccttctaaagcacggaagatggttcttggaggctaatgtaggcgtaggtggagaatcaatttccaaaggaggaaactctgctaacggtcttggctttggcttcggacctggatactcttatttcttgacaccgaacatcggactagagggagttgttaaatatgatgcaaatgcgggctttggtaatggaggttatacccacaatattaaatttggtttagggctcaacatctttatttctactgcaaaggcagaagaaatcattaaaaatgtaaaataa

(19) United States
(12) Patent Application Publication
(10) Pub. No.: US 2025/0000941 A1
(43) Pub. Date: Jan. 2, 2025

(54) Recombinant Protein For Inducing Cytoplasmic Vacuolization Cell Death And Use Thereof

(71) Applicant: Shandong University, Jinan (CN)

(72) Inventors: Xiaopeng QI, Jinan (CN); Rudi MAO, Jinan (CN); Tao XU, Jinan (CN); Hongwei PAN, Jinan (CN); Can HUANG, Jinan (CN); Zhen LI, Jinan (CN)

(21) Appl. No.: 18/412,867

(22) Filed: Jan. 15, 2024

(30) Foreign Application Priority Data
Jun. 28, 2023 (CN) 202310769419.2

(51) Int. Cl. A61K 38/17; A61P 35/00

(52) U.S. Cl. CPC A61K 38/1709; A61P 35/00

(57) ABSTRACT

The present disclosure provides a recombinant protein for inducing cytoplasmic vacuolization cell death and use thereof, and belongs to the technical field of biomedicine. The present disclosure provides a recombinant protein for inducing cell death, where the recombinant protein is at least one selected from the group consisting of PorV, Lipocalin, and β-barrel; and the PorV has an amino acid sequence shown in SEQ ID NO: 1, the Lipocalin has an amino acid sequence shown in SEQ ID NO: 2, and the B-barrel has an amino acid sequence shown in SEQ ID NO: 3. In the present disclosure, the recombinant protein induces the formation of cytoplasmic vacuolization in various cells, thereby inducing the vacuolar cell death.

### CROSS REFERENCE TO RELATED APPLICATION

This patent application claims the benefit and priority of Chinese Patent Application No. 202310769419.2, filed with the China National Intellectual Property Administration on Jun. 28, 2023, the disclosure of which is incorporated by reference herein in its entirety as part of the present application.

### REFERENCE TO SEQUENCE LISTING

This application contains references to amino acid sequences and/or nucleic acid sequences which have been submitted concurrently herewith as the sequence listing.xml file entitled “000065us_SequenceListing.xml”, file size 10,329 bytes, created on Jan. 9, 2024. The aforementioned sequence listing is hereby incorporated by reference in its entirety pursuant to 37 C.F.R. § 1.52 (e) (5).

### TECHNICAL FIELD

The present disclosure belongs to the technical field of biomedicine, and specifically relates to a recombinant protein for inducing cytoplasmic vacuolization cell death and use thereof.

### BACKGROUND

Cells death shows various approaches depending on the cellular context and triggering stimulus. Programmed cell death (PCD) is necessary for normal development and maintenance of tissue homeostasis and for the elimination of damaged, infected, or senescent cells. The most classic types of PCD that have been discovered so far include apoptosis, necroptosis, and pyroptosis. In recent years, more PCDs have been discovered, such as ferroptosis, lysosome-dependent cell death (LCD), autophagy-dependent cell death (ACD), cuproptosis, and cytoplasmic vacuolization cell death (vacuolar PCD). PCD removes useless cells as well as infected or potentially-neoplastic cells and plays an important role in homeostasis, host defense against pathogens, cancer and a range of other pathologies. Increasing drugs are being developed to treat infections, aging, cancer and other related diseases by targeting and regulating different PCD approaches. Novel death-inducing drugs for cancer cells demonstrate important application prospects in the treatment of drug-resistant cancer cells and apoptosis-resistant cancer cells.

However, there are currently no records or reports in the prior art regarding use of PorV, Lipocalin, and β-barrel molecules in the preparation of drugs that induce cell death.

### SUMMARY

An objective of the present disclosure is to provide a recombinant protein for inducing cytoplasmic vacuolization cell death and use thereof. The recombinant protein can induce the formation of cytoplasmic vacuolization and can be used as a therapeutic target for cancers, autoimmune diseases, and chronic inflammations.

The present disclosure provides a recombinant protein for inducing cell death, where the recombinant protein is at least one selected from the group consisting of PorV, Lipocalin, and β-barrel; and

the PorV has an amino acid sequence shown in SEQ ID NO: 1, the Lipocalin has an amino acid sequence shown in SEQ ID NO: 2, and the β-barrel has an amino acid sequence shown in SEQ ID NO: 3.

Preferably, a cell is selected from the group consisting of a tumor cell and a normal immune cell.

Preferably, a cell-induced disease is selected from the group consisting of a cancer, an autoimmune disease, and a chronic inflammation.

Preferably, the cell death includes cytoplasmic vacuolization cell death.

The present disclosure further provides a gene encoding the recombinant protein.

Preferably, a gene encoding the PorV has a nucleotide sequence shown in SEQ ID NO: 4, a gene encoding the Lipocalin has a nucleotide sequence shown in SEQ ID NO: 5, and a gene encoding the β-barrel has a nucleotide sequence shown in SEQ ID NO: 6.

The present disclosure further provides use of the recombinant protein or the gene as a therapeutic target in production of a preparation for inducing the cell death.

Preferably, the therapeutic target further includes a drug with a therapeutic effect on the cell.

Preferably, a process of inducing the cell death includes inducing the cytoplasmic vacuolization cell death.

The present disclosure further provides a drug for inducing cell death, including the recombinant protein and a pharmaceutically acceptable auxiliary material.

Beneficial effects: the present disclosure provides a recombinant protein for inducing cell death, where the recombinant protein is at least one selected from the group consisting of PorV, Lipocalin, and β-barrel. In the present disclosure, the recombinant protein can induce a variety of cells and normal immune cells, such as cancer cells, autoimmune disease cells, and chronic inflammation-infected cells, to produce cytoplasmic vacuolization, thereby inducing the cell death. It is confirmed for the first time that the recombinant protein molecule shows new use in production of a preparation for inducing cytoplasmic vacuolization. The recombinant protein can be used as a therapeutic target for a cancer, an autoimmune disease, and a chronic inflammation, exhibiting a potential pharmaceutical value as the therapeutic target during treatment of cancers and persistent infections.

### BRIEF DESCRIPTION OF THE DRAWINGS

FIGS. 1A-B show the cloning results of recombinant Lipocalin, β-barrel, and PorV proteins (FIG. 1A) and the formation of cytoplasmic vacuolization induced by transfection of Lipocalin, β-barrel, and PorV proteins in macrophages (FIG. 1B);

FIG. 2 shows the formation of cytoplasmic vacuolization induced in cells 12 h after co-transfection of Lipocalin and β-barrel proteins as well as combined transfection of Lipocalin, β-barrel, and PorV proteins; and

FIG. 3 shows the formation of cytoplasmic vacuolization in liver cancer cells (Huh-7) induced by transfection of Lipocalin, β-barrel, and PorV proteins.

### DETAILED DESCRIPTION OF THE EMBODIMENTS

The present disclosure provides a recombinant protein for inducing cell death, where the recombinant protein is at least one selected from the group consisting of PorV, Lipocalin, and β-barrel; and
the PorV has an amino acid sequence shown in SEQ ID NO: 1: MNLKSKLFVGLGLGLSLSAAAQEVKPVLTGAPFLRITPDARAAGMGDQGVATSTDAFSQF WNAAKYPFSKTTSAVAVNYTPYLSKLTSDVFLLYASYHQFLGQEERSTISASINYFNMGEVE LNQLSGTQIVTTGKVKPNEFSVDVAYGLKLSDVYSMAVTGRYIRSDFGTFGDNSTIKPANTF AVDVSGFYQSEKHSSFGDYEGTARAGWAIQNLGPKLDYSGDENSRSYLPTTLRLGAGYDLY LDDMNKVGLSLEASKLLVPGSQLNNSDPANPKYEVPNVGVVEGIGKSFSNKKSVMLSGAV EYSYDDAFAVRGGYFHESDEQGGRRFATVGVGFKYQSFGLDISYLINTSKTNTALDNTLRFG LTWNIGGESSNANN;
the Lipocalin has an amino acid sequence shown in SEQ ID NO: 2: MKNLFLIGALGSALAVASCSPTKIAQNKKNKTDFAKLKGNWQITSVDYDHNYRVKPFDEGI DAQCFVGSQWKLVPNNYSGTYALLGEGRCPSFSRVIKFELVDGAEFKFKKIVEGTKAKDNI AGYSLYLLDQTDTSFSLEQNVSGVRIVYNFQKTN; and
the β-barrel has an amino acid sequence shown in SEQ ID NO: 3: MTKLISTALIGLSALVSAQVSFAGKAHLLFPTSSGSWKSLKDAGLKAYDKKGSNSVGYNVG VSAKVDLPGAFYVMPELYFTSFKNEFTDENTKTSLKAKSNRIDVPVLVGYKLLGDTASLFL GPVASYNLSTKESYQNFKEHATKDFTVGYQFGAQVQLSDFIINARYEGAFSNDQRNFINKH VVSGQEYEVQYDARPSLFMVGVGYKF.

In the present disclosure, the recombinant proteins PorV, Lipocalin, and β-barrel are preferably obtained by cloning a target gene from a genome of endocarditis bacteria into an expression vector, and purification. Preferably, cloned genes (SEQ ID NO: 4, SEQ ID NO: 5, and SEQ ID NO: 6) are inserted into a PGEX-6P-2 vector, and a constructed recombinant vector PGEX-6P-2-PorV is transformed into E. coli Rosetta (DE3) pLysS for induced expression, and purified according to a purification tag.

In the present disclosure, the recombinant protein is an inducer of various cell deaths, especially an inducer of cytoplasmic vacuolization cell death. A cell includes a cancer cell and/or a normal immune cell, and can serve as a therapeutic target for a cancer, an autoimmune disease, and a chronic inflammation.

The present disclosure further provides a gene encoding the recombinant protein.

In the present disclosure, a gene encoding the PorV has a nucleotide sequence preferably shown in SEQ ID NO: 4: ATGAACTTAAAATCAAAATTATTTGTAGGACTTGGGCTTGGGCTTAGCCTTTCGGCAGCA GCTCAAGAAGTAAAACCCGTACTTACAGGGGCTCCTTTCCTCAGAATAACTCCAGATGC AAGAGCAGCAGGTATGGGGGACCAAGGGGTAGCAACCTCTACAGATGCATTCTCTCAGT TTTGGAATGCCGCTAAGTACCCATTCAGTAAAACTACCTCTGCTGTAGCAGTAAACTATA CGCCTTATCTCAGCAAACTTACAAGCGATGTATTCCTCCTCTATGCTTCTTATCATCAGTTT CTGGGGCAGGAAGAGCGATCTACCATCTCTGCAAGTATCAATTATTTTAATATGGGAGAG GTGGAACTCAATCAACTGTCAGGCACACAAATCGTAACTACAGGTAAGGTAAAGCCTAA TGAATTTTCTGTAGATGTAGCCTATGGGCTGAAACTCTCAGATGTATACTCTATGGCAGTT ACAGGTAGATACATCCGCTCGGATTTCGGTACCTTTGGAGACAACAGCACTATAAAACCT GCCAATACTTTCGCAGTAGATGTTTCAGGTTTCTATCAGTCTGAAAAGCACTCCAGCTTT GGAGACTACGAAGGTACAGCAAGAGCGGGCTGGGCTATTCAGAACCTTGGACCAAAAT TGGACTATTCAGGAGATGAAAACTCTCGTTCCTATCTGCCAACTACACTCAGATTAGGTG CTGGCTATGATTTATATCTCGATGATATGAATAAAGTAGGGCTTTCCCTTGAAGCATCAAA ACTCCTCGTTCCAGGCTCTCAGCTAAATAATTCCGATCCTGCCAACCCTAAATACGAAGT TCCTAATGTAGGTGTAGTAGAGGGTATAGGTAAATCTTTCAGCAATAAAAAAAGCGTAAT GCTAAGCGGTGCTGTGGAGTACTCCTATGATGATGCTTTTGCTGTGCGTGGTGGTTACTT CCACGAGAGCGATGAGCAGGGTGGCAGACGCTTCGCTACCGTAGGGGTGGGCTTTAAAT ATCAGTCTTTCGGGCTGGATATCTCCTACCTCATCAATACTTCAAAGACAAATACTGCTCT TGACAATACCCTCCGTTTCGGTCTTACCTGGAATATTGGTGGTGAGTCTTCCAACGCCAA TAATTAG;
a gene encoding the Lipocalin has a nucleotide sequence preferably shown in SEQ ID NO: 5: ATGAAAAACTTATTTCTAATAGGAGCTTTGGGCTCTGCGTTGGCGGTAGCATCTTGCTCG CCTACAAAAATAGCACAGAATAAAAAGAATAAGACAGACTTTGCAAAGCTCAAAGGCA ATTGGCAGATTACAAGCGTGGATTATGACCATAATTATCGTGTAAAACCTTTTGATGAAG GCATTGATGCGCAGTGCTTCGTCGGTAGTCAATGGAAATTGGTGCCCAATAACTACTCTG GTACCTATGCGCTCCTTGGTGAAGGCCGTTGCCCATCGTTTTCCAGAGTTATAAAGTTTG AGCTTGTGGATGGTGCAGAGTTTAAGTTTAAAAAAATAGTAGAGGGAACCAAGGCTAAG GACAATATAGCAGGATACTCTCTCTATCTCCTCGACCAAACCGATACTTCCTTTAGCTTAG AACAGAATGTAAGCGGTGTAAGAATCGTTTATAACTTCCAAAAGACAAATTAG; and
a gene encoding the β-barrel has a nucleotide sequence preferably shown in SEQ ID NO: 6: ATGAGAAAGATTATTCTTGGAGCTATAGTATTAGGAGCAGGATTATTCTCCAATGCTAATG CACAAATCCAAAAAGGTAATTTGATAATTGGTGGAGACATCTTCGGTGCTAATTTTGGAC TGAACAAAGGTGGAGGATATAACTTCAATCTTCAACCGAAAGCAGCTTATTTTGTAGAA GATAATGTAGCAGTCGGTGGATATGCAGACTTAGGTTTCAGTGGCTCGAACGGAGGACA AACAAAATTCACTTATACAGTAGGAGCTTTGGGAAGATATTACTTATCACCAGGACAAGG TGGCGTAGATAACCTTCTAAAGCACGGAAGATGGTTCTTGGAGGCTAATGTAGGCGTAG GTGGAGAATCAATTTCCAAAGGAGGAAACTCTGCTAACGGTCTTGGCTTTGGCTTCGGA CCTGGATACTCTTATTTCTTGACACCGAACATCGGACTAGAGGGAGTTGTTAAATATGAT GCAAATGCGGGCTTTGGTAATGGAGGTTATACCCACAATATTAAATTTGGTTTAGGGCTC AACATCTTTATTTCTACTGCAAAGGCAGAAGAAATCATTAAAAATGTAAAATAA.

In the present disclosure, the genes shown in SEQ ID NO: 4, SEQ ID NO: 5, and SEQ ID NO: 6 are preferably subjected to induced expression using a prokaryotic expression vector; preferably, the genes are inserted into BamHI and XhoI sites of the PGEX-6P-2 vector.

The present disclosure further provides use of the recombinant protein or the gene as a therapeutic target in production of a preparation for inducing the cell death.

In the present disclosure, the recombinant protein can be used as a separate therapeutic target, or a combination of at least two different recombinant proteins can be used as the therapeutic target, or at least one of the recombinant proteins can be used in combination with other therapeutic drugs for corresponding diseases.

The present disclosure further provides a drug for inducing cell death, including the recombinant protein and a pharmaceutically acceptable auxiliary material. In the present disclosure, in addition to the recombinant protein, active ingredients of the drug may also include other drugs with therapeutic effects.

In order to further illustrate the present disclosure, the recombinant protein for inducing cytoplasmic vacuolization cell death and the use thereof provided by the present disclosure are described in detail below in connection with examples, but these examples should not be understood as limiting the claimed scope of the present disclosure.

### Example 1

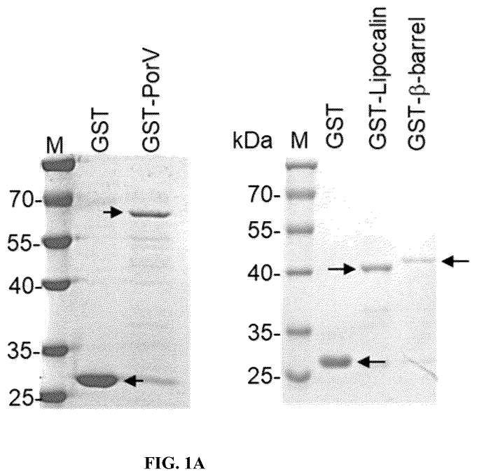

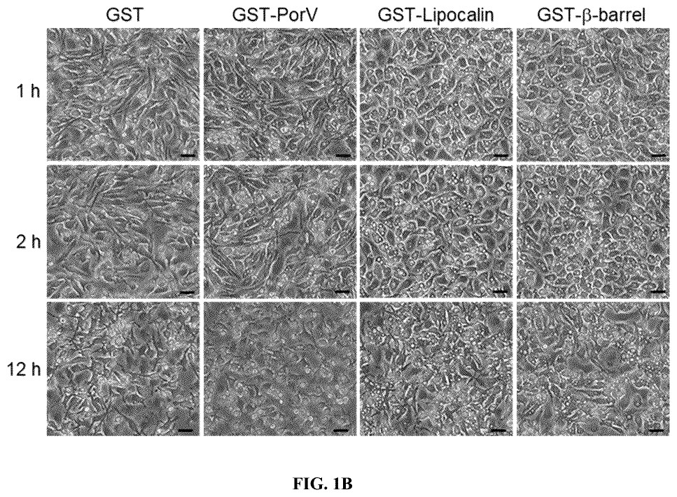

1) The genes shown in SEQ ID NO: 4, SEQ ID NO: 5, and SEQ ID NO: 6 were inserted between the BamHI and XhoI sites of the PGEX-6P-2 vector separately to construct a recombinant plasmid; the recombinant plasmid was used to transform competent cells of an E. coli expression strain RDP (Rosetta (DE3) pLysS).
2) A single colony or multiple colonies were selected to allow culture overnight at 37° C. with shaking; the next day, a resulting bacterial solution was transferred to 1 L of a medium (to allow culture at 37° C. for about 2 h to 3 h until OD₆₀₀=0.5-0.8).
3) The cells were induced with IPTG (0.3 mM), and cultured at 16° C. overnight until OD₆₀₀>1.0.
4) The bacterial cells were collected, the bacterial solution was resuspended in a lysis solution (50 mM Tris-HCl, pH=7.4+100 mM NaCl+1 mM EDTA+1 mM DTT+PIC), and cells were disrupted by sonication in an ice-water bath.
5) The bacterial solution was centrifuged at a high speed (12,000 g for 30 min) at 4° C. to collect a protein supernatant.
6) The protein supernatant was incubated with glutathione agarose for 2 h, and then balanced and purified with a reduced glutathione buffer. A protein size measured by SDS-PAGE was consistent with the prediction result (FIG. 1A).

### Example 2

1) Preparation of primary mouse BMDM cells: adult C57/BL mice of appropriate age (such as 4 to 6 weeks old) were euthanized; mouse femur and tibia were separated under sterile conditions and placed in a PBS buffer; the muscles, cartilage, and epiphysis on the bone surface were scraped with scalpel, and both ends of the bone were cut to expose the medullary cavity; the medullary cavity was rinsed with DMEM-F12 medium by a 1 mL syringe to collect cells; the cells were pipetted evenly, counted, and adjusted to a cell density of (1-2) Million/mL, and the cells were inoculated in a DMEM-F12 complete medium with 20 ng/ml of M-CSF growth factor in a cell culture plate; the cells were cultured statically in an incubator with 5% CO2 and 37° C. for 5 d, and then adherent cells were collected for corresponding experiments.
2) Culture of endocarditis bacteria: the endocarditis bacteria were activated on a Columbia blood agar until the bacteria grew typical colonies, then cultured on the blood agar at 37° C. with 5% CO₂.
3) Expression and purification of Lipocalin, β-barrel, and PorV proteins: according to the method of Example 1, a control PGEX-6P-2 vector and the PGEX-6P-2-PorV construct were transformed into E. coli Rosetta (DE3) pLysS to allow expression. The expressed GST-Lipocalin, GST-β-barrel, and GST-PorV and a control GST protein were incubated with glutathione agarose for 2 h separately, and then balanced and purified with a reduced glutathione buffer. After purification, the GST-Lipocalin, GST-β-barrel, and GST-PorV and the control GST protein were transfected into cells using an Xfect™ protein transfection reagent.

As shown in FIG. 1B, Lipocalin, β-barrel and PorV proteins induced the formation of cell vacuoles 1 h, 2 h and 12 h after the transfection.

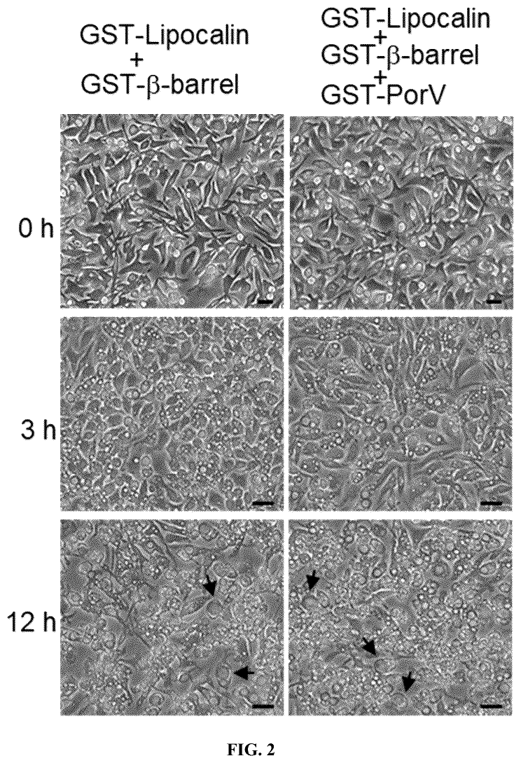

As shown in FIG. 2, cell vacuoles were induced 3 h and 12 h after co-transfection of GST-Lipocalin and GST-β-barrel or a combination of three proteins: GST-Lipocalin, GST-β-barrel, and GST-PorV.

### Example 3

1) Culture of Huh-7 cells: the Huh-7 cells as liver cancer cells were inoculated and cultured using DMEM complete medium in a cell culture plate in an incubator with 5% CO₂ at 37° C.

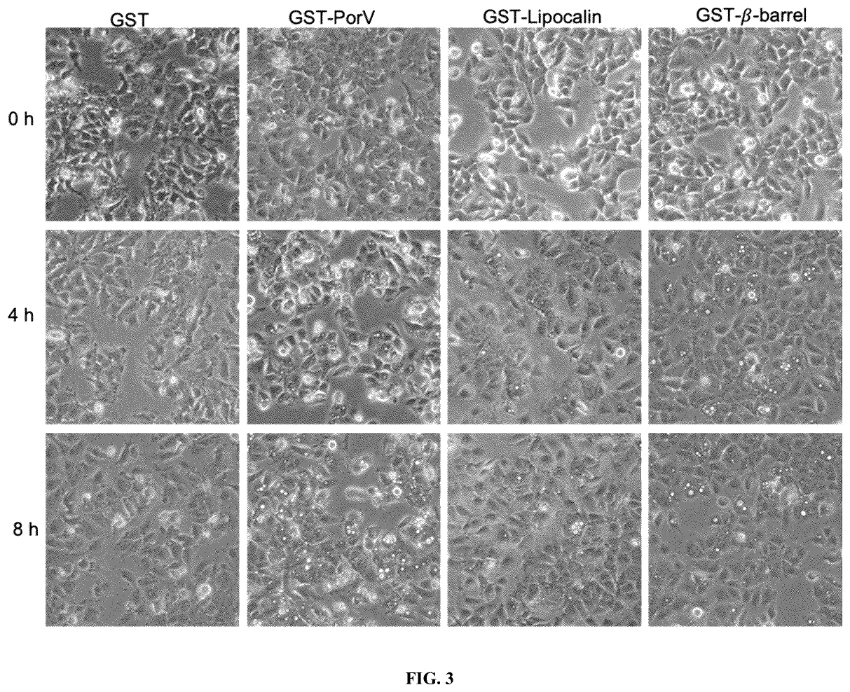

As shown in FIG. 3, the transfection of Lipocalin, β-barrel, and PorV proteins resulted in the formation of cell vacuoles at 4 h and 8 h. The above results indicated that the Lipocalin, β-barrel, and PorV were important virulence factors in endocarditis-induced cytoplasmic vacuolation.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A recombinant protein for inducing cell death, wherein the recombinant protein is at least one selected from the group consisting of PorV, Lipocalin, and β-barrel; and
the PorV has an amino acid sequence shown in SEQ ID NO: 1, the Lipocalin has an amino acid sequence shown in SEQ ID NO: 2, and the β-barrel has an amino acid sequence shown in SEQ ID NO: 3.

2. The recombinant protein according to claim 1, wherein a cell is selected from the group consisting of a tumor cell and a normal immune cell.

3. The recombinant protein according to claim 2, wherein a cell-induced disease is selected from the group consisting of a cancer, an autoimmune disease, and a chronic inflammation.

4. The recombinant protein according to claim 1, wherein the cell death comprises cytoplasmic vacuolization cell death.

5. The recombinant protein according to claim 2, wherein the cell death comprises cytoplasmic vacuolization cell death.

6. A gene encoding the recombinant protein according to claim 1.

7. A gene encoding the recombinant protein according to claim 2.

8. A gene encoding the recombinant protein according to claim 3.

9. A gene encoding the recombinant protein according to claim 4.

10. A gene encoding the recombinant protein according to claim 5.

11. The gene according to claim 6, wherein a gene encoding the PorV has a nucleotide sequence shown in SEQ ID NO: 4, a gene encoding the Lipocalin has a nucleotide sequence shown in SEQ ID NO: 5, and a gene encoding the β-barrel has a nucleotide sequence shown in SEQ ID NO: 6.

12. A method for production of a preparation for inducing cell death, comprising: administrating the recombinant protein according to claim 1.

13. A method for production of a preparation for inducing cell death, comprising: administrating the gene according to claim 6 as a therapeutic target.

14. The method according to claim 12, wherein the therapeutic target further comprises a drug with a therapeutic effect on the cell.

15. The method according to claim 12, wherein a process of inducing the cell death comprises inducing the cytoplasmic vacuolization cell death.

16. A drug for inducing cell death, comprising the recombinant protein according to claim 1 and a pharmaceutically acceptable auxiliary material.

17. A drug for inducing cell death, comprising the recombinant protein according to claim 2 and a pharmaceutically acceptable auxiliary material.

18. A drug for inducing cell death, comprising the recombinant protein according to claim 3 and a pharmaceutically acceptable auxiliary material.

19. A drug for inducing cell death, comprising the recombinant protein according to claim 4 and a pharmaceutically acceptable auxiliary material.

20. A drug for inducing cell death, comprising the recombinant protein according to claim 5 and a pharmaceutically acceptable auxiliary material.
